(19) United States
(12) Patent Application Publication
(10) Pub. No.: US 2005/0168570 A1
(43) Pub. Date: Aug. 4, 2005

(54) Flat panel display and driving method thereof

(76) Inventors: Hyun-Jae Kim, Seoul (KR); Oh-Kyung Kwon, Seoul (KR)

Correspondence Address: DLA PIPER RUDNICK GRAY CARY US, LLP, 2000 UNIVERSITY AVENUE, E. PALO ALTO, CA, 94303-2248, US

(21) Appl. No.: 10/508,593

(22) PCT Filed: Sep. 18, 2002
(86) PCT No.: PCT/KR02/01772

(30) Foreign Application Priority Data
Apr. 19, 2002 (KR) 2002/21541

(51) Int. Cl. A61B 1/04

(52) U.S. Cl. 348/71

(57) ABSTRACT

According to the present invention, a flat panel display is provided, which includes a display panel where a plurality of pixels are formed. One pixel includes a first and a second signal lines extending in any one direction and a pixel circuit connected to the first and the second signal lines, and the first and the second signal lines intersect with each other. The pixel circuit includes a plurality of storages, a plurality of first and second switching elements connected to the storages, respectively, and a display cell. The storages store data transmitted through the first signal during predetermined time. The first switching elements send data transmitted through the first signal line to the respective storages in response to a signal transmitted through the second signal line. The second switching elements are sequentially driven during one frame to transmit the data stored in the storages to the display cell. The display cell displays image of the pixel according to the data stored in the storages.

### BACKGROUND OF THE INVENTION

(a) Field of the Invention

The present invention relates to a flat panel display and a driving method thereof, and more particularly to a flat panel display including a plurality of pixels having memory circuits therein and a driving method thereof.

(b) Description of the Related Art

In recent year, as personal computers and television sets become light-weighted and slim, so a display is required to be the same. In order to fulfill such requirements, flat panel displays such as a liquid crystal display (“LCD”) instead of a cathode ray tube (“CRT”) are developed.

These flat panel displays include a liquid crystal display (“LCD”), a field emission display (“FED”), a electroluminescent display, and a plasma display panel (“PDP”).

These flat panel displays have a problem of implementing a variety of grays. The LCD, which includes an upper panel with a common electrode and color filters, a lower panel with thin film transistors (“TFTs”) and pixel electrodes, and a liquid crystal layer disposed therebetween, applies different electric potentials to the pixel electrodes and the common electrode to generate electric field to change the arrangement of liquid crystal molecules, thereby controlling the transmittance of light to implement a variety of grays.

The electroluminescent display implements a variety of grays by controlling applied data voltages in several grades in a predetermined range since currents corresponding to the data voltages applied to pixel circuits are applied to electroluminescent devices and the electroluminescent devices emit light depending on the applied currents.

The PDP implements 2^{N} grays by dividing one frame into N subframes, each subframe including an addressing period for determining whether to implement grays and a display period for implementing grays, and then discriminating display time of each sub-frame by exponent of 2.

Although the above-described driving method for the PDP is applicable to a gray implementation of a flat panel display including pixels driven in active matrix type, there is a problem that the addressing in each subframe yields increased power consumption, the addressing period reduces the display period, and the display period is limited to exponent of 2.

### SUMMARY OF THE INVENTION

Considering this problem, a motivation of the present invention is to decrease the power consumption in gray implementation.

The present invention provides a plurality of storages at a pixel and sequentially drives a display cell with data stored in the storages, thereby accomplishing the motivation.

According to an aspect of the present invention, a flat panel display is provided, which includes a display panel provided with a plurality of pixels. A pixel includes first and second signal lines extending in respective directions and intersecting each other and a pixel circuit connected to the first and the second signal lines. The pixel circuit includes a plurality of storages, a plurality of first and second switching elements connected to the storages, and a display cell. The storages store data from the first signal line during a predetermined time. The first switching elements transmit data from the first signal line to the respective storages in response to a signal from the second signal line. The second switching elements are sequentially driven during a frame to transmit the data stored in the storages to the display cell. The display cell displays image for the pixel according to the data stored in the storages.

The pixel circuit may further include a plurality of inverting switching elements for inverting the data stored in the storages to be applied to the display cell.

The data stored in the storages preferably have a first value making the display cell represent white gray and a second value making the display cell represent black gray.

The pixel circuit according to the first aspect of the present invention may further include a third switching element connected between one of the first signal lines and the display cell, and the third switching element transmits data representing a variety of grays from the first signal line to the display cell in response to a signal from the second signal line.

The first signal line may include a plurality of signal lines connected to the first switching elements, respectively. Alternatively, the first signal line may include one signal line and the number of the second signal lines may equal to the number of the first switching elements. Alternatively, the first signal line and the second signal line include a plurality of signal lines, respectively, and the first switching elements may correspond to one of the first signal lines and the second switching elements may correspond to one of the second signal lines, respectively.

The address time is a predetermined time shorter than one frame or equal to or longer than one frame.

According to a second aspect of the present invention, a method of driving a flat panel display including a plurality of pixels having display cells is provided. According to the method, data are stored in a plurality of storages during a predetermined address time, respectively. Next, one frame or a portion of one frame is divided into a plurality of sub-frames and the respective display cells are sequentially driven with the data stored in the storages during sub-frames, thereby displaying gray.

When data representing still images are inputted, data are stored in the storages, and when data representing moving images are inputted, the display cells may be directly driven.

Preferably, the data stored in the storages and inverted data are alternately applied to the display cells, thereby driving the display cells.

### BRIEF DESCRIPTION OF THE DRAWINGS

FIG. 1 illustrates an LCD according to a first embodiment of the present invention;

FIG. 2 illustrates a single pixel circuit of an LCD according to a first embodiment of the present invention;

FIG. 3 shows driving waveform for implementing gray in an LCD according to a first embodiment of the present invention;

FIG. 4 illustrates a single pixel circuit of an LCD according to a second embodiment of the present invention;

FIG. 5 illustrates an LCD according to a third embodiment of the present invention;

FIG. 6 and FIG. 7 illustrate single pixel circuits of LCDs according to third and fourth embodiments of the present invention;

FIG. 8 illustrates an LCD according to a fifth embodiment of the present invention; and

FIG. 9 and FIG. 10 illustrate single pixel circuits of LCD according to fifth and sixth embodiments of the present invention.

### DETAILED DESCRIPTION OF PREFERRED EMBODIMENTS

Now, flat panel displays and driving methods thereof will be described in detail with reference to accompanying drawings.

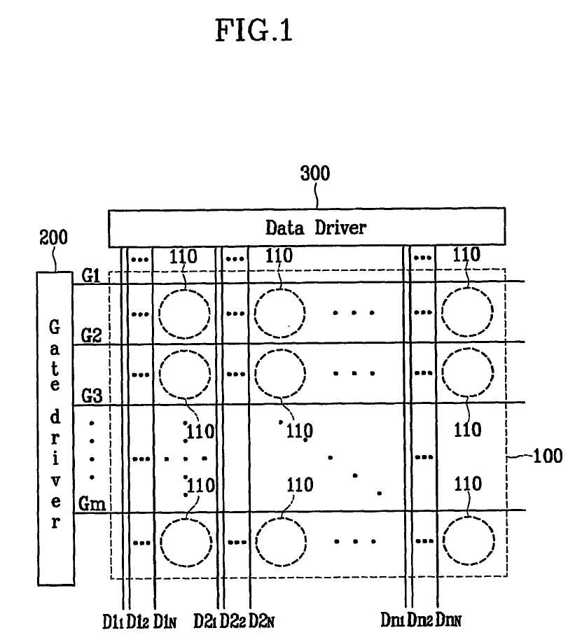

First, an LCD and a driving method thereof according to a first embodiment of the present invention will be described with reference to FIG. 1 to FIG. 3.

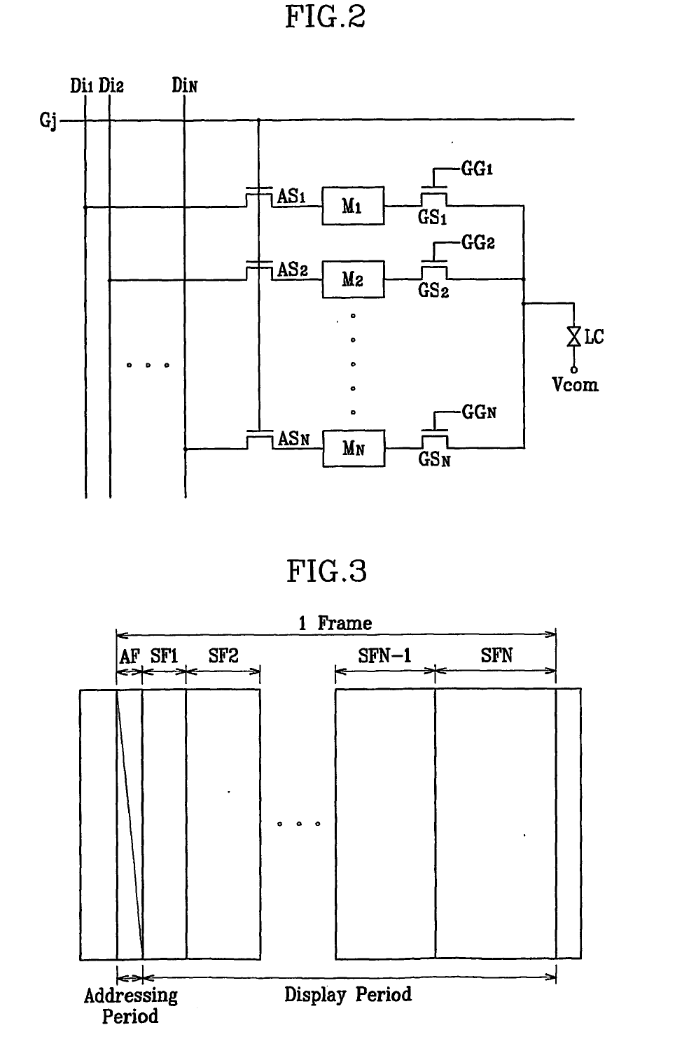

FIG. 1 shows an LCD according to a first embodiment of the present invention, and FIG. 2 shows a single pixel circuit of an LCD according to a first embodiment of the present invention. FIG. 3 shows a driving waveform for implementing gray in an LCD according to a first embodiment of the present invention.

As shown in FIG. 1, an LCD according to the first embodiment of the present invention includes a liquid crystal panel 100, a gate driver 200, and a data driver 300.

The gate driver 200 applies gate signals for selecting pixels of the liquid crystal panel 100 to the liquid crystal panel 100 through a plurality of gate lines G1-Gm.

The data driver 300 applies signals representing images to the liquid crystal panel 100 through a plurality of groups of data lines D1-Dn, and one data line group Di for one column (i-th column) includes N signal lines Di₁-Di_{N}. The data voltages applied to the N signal lines Di₁-Di_{N} represent white or black gray.

The liquid crystal panel 100 includes a plurality of pixel circuits 110 arranged in a matrix, and each pixel circuit 110 is formed in an area defined by two neighboring gate lines G1-Gm and two neighboring data line groups D1-Dn, which is connected to adjacent one gate line and one data line group.

Now, referring to FIG. 2, a single pixel circuit 110 connected to i-th data lines Di₁-Di_{N} and a j-th gate line Gj will be described in detail.

As shown in FIG. 2, a single pixel circuit 110 of an LCD according to the first embodiment of the present invention includes N memory circuits M₁-M_{N}, N addressing switching elements AS₁-AS_{N}, N gray switching elements GS₁-GS_{N}, and a liquid crystal cell LC.

Each address switching element AS₁-AS_{N} is connected between a data line Di₁-Di_{N} and a memory circuit M₁-M_{N}, and stores data in the respective memory circuit M₁-M_{N} in response to the gate signals from the gate driver 200.

Each gray switching element GS₁-GS_{N} is connected between a memory circuit M₁-M_{N} and the liquid crystal cell LC, and drives the liquid crystal cell LC with the data stored in the memory circuit M₁-M_{N} in response to driving signals from an external device.

A common electrode, which is one terminal of the liquid crystal cell LC, is supplied with a common electrode voltage Vcom, and the common electrode voltage Vcom displays a gray together with a data voltage applied to a pixel electrode, which is the other terminal of the liquid crystal cell LC.

The first embodiment of the present invention divides a single frame into an addressing frame AF and N sub-frames SF1-SFN. In detail, the data are stored in the memory circuits M₁-M_{N} during the addressing period, and then, the liquid crystal cell LC is driven with the data stored in the memory circuits by sequentially driving the gray switching elements during the sub-frame intervals SF1-SFN.

As described above, when the liquid crystal cell LC is driven with N sub-frames, it is possible to display a 2^{N} gray image. The period of sub-frames may be divided into exponent of 2 like an ADS type PDP, or, without being divided into exponent of 2, it may be determined through signal processing in consideration of a gamma correction and image quality improvement.

Now, referring to FIG. 3, a driving method of the LCD according to the first embodiment of the present invention will be described.

As shown in FIG. 3, the first embodiment of the present invention drives dividing a frame into an addressing frame AF and N sub-frames SF1-SFN.

During the addressing frame AF, a gate signal for selecting pixel circuits of the rows is applied to one of the gate lines GS₁-GS_{N}. When the gate signal is applied to j-th gate line Gj, the data voltages are stored in the respective memory circuits M₁-M_{N} connected to the j-th gate line Gj. That is, the addressing switching elements AS₁-AS_{N} of the respective pixel circuits connected to the j-th gate line are turned on, and thereby, the data voltages applied through the data lines Dil-DiN from the data driver 300 are stored in the memory circuits M₁-M_{N}.

After the data voltages are stored in the memory circuits during the addressing frame AF, the liquid crystal cell LC is sequentially driven with the data stored in the memory circuits during the sub-frames SF1-SFN.

In detail, the gray switching element GS₁ of the pixel circuit drives the liquid crystal cell LC with the data voltage stored in the memory circuit M₁ during the first sub-frame SF1 in response to a GG₁ signal, and the gray switching element GS₂ of the pixel circuit drives the liquid crystal cell LC with the data voltage stored in the memory circuit M2 during the second sub-frame SF2 in response to a GG₂ signal. In this manner, the liquid crystal cell LC is driven with the data voltages stored in the memory circuits M₁-M_{N} of the pixel circuit during the sub-frames SF1-SFN.

For example, in case of the LCD in a normally white mode, the data voltage stored in the memory circuit, if having the same value as the common electrode voltage Vcom, represents a white gray, while the data voltage represents a black gray if it has a value different from the common electrode voltage Vcom. In this case, the gray is determined by ratio of time for representing the white gray and time for displaying the black gray. Thus, the first embodiment of the present invention implements 2^{N} grays since there are N memory circuits in one pixel.

As described above, the first embodiment of the present invention, when displaying still images, stores the data in the memory circuits at first, and thereafter, drives the liquid crystal using the data stored in the memory circuits without re-applying the data voltages from the data driver. In case of displaying moving images, new data are stored in the memory circuits during each addressing frame, the liquid crystal is driven using such data.

In the meantime, when a gray is displayed in one frame and another gray is displayed in the next frame, DC bias applied across the liquid crystal cell LC may deteriorate characteristics of the liquid crystal. To prevent it, generally, an inverting switching element (not shown) for applying inverted data and inverted common electrode voltage to the liquid crystal cell LC may be employed in the pixel circuit. It is apparent that the above-described inverting switching element is also applicable to other embodiments described below.

Although the first embodiment of the present invention displays grays of all images using the memory circuits, grays of still images may be displayed using the memory circuit while grays of moving images may be displayed by directly driving the liquid crystal cell without using the memory circuits.

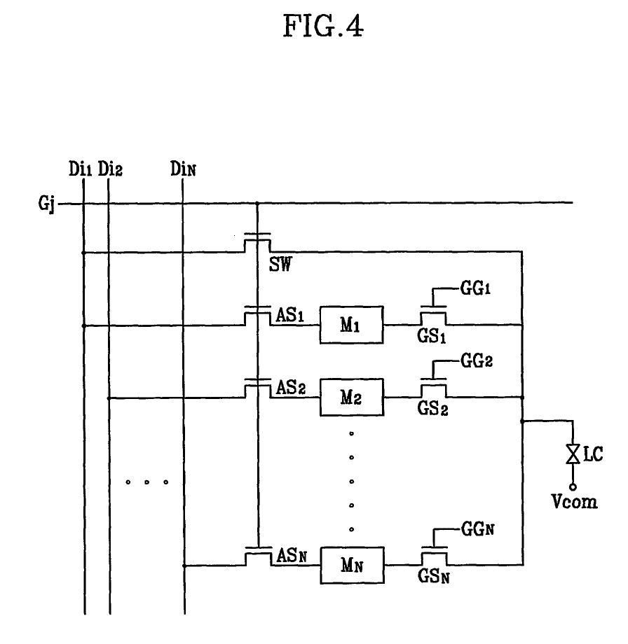

Referring to FIG. 4, an embodiment using such a driving method will be described in detail.

FIG. 4 shows a single pixel of an LCD according to a second embodiment of the present invention.

As shown in FIG. 4, an LCD according to the second embodiment of the present invention has substantially the same configuration as that according to the first embodiment except that a pixel circuit 110 further includes an analog switching element SW.

The pixel circuit 110 connected to i-th data lines Di and j-th gate line Gj will be described in detail. This pixel circuit 110 further includes an analog switching element SW connected between one signal line (e.g., Di₁) of data lines Di₁-Di_{N} and a liquid crystal cell LC.

In case of displaying still images, the data are once stored in the memory circuits like the first embodiment of the present invention, and the liquid crystal is driven using the stored data. In case of displaying moving images, unlike the first embodiment of the present invention, addressing switching elements AS₁-AS_{N} and gray switching elements GS₁-GS_{N} are turned off and the analog switching element SW is turned on. Then, the liquid crystal cell LC is driven with analog data voltage applied through the data line Di₁. The analog data voltage represents a variety of grays as well as white and black grays.

The first and the second embodiments of the present invention store the data in the respective memory circuits by dividing one data line into a plurality of signal lines. Alternately, the data are stored in the memory circuit by dividing one gate line into a plurality of signal lines.

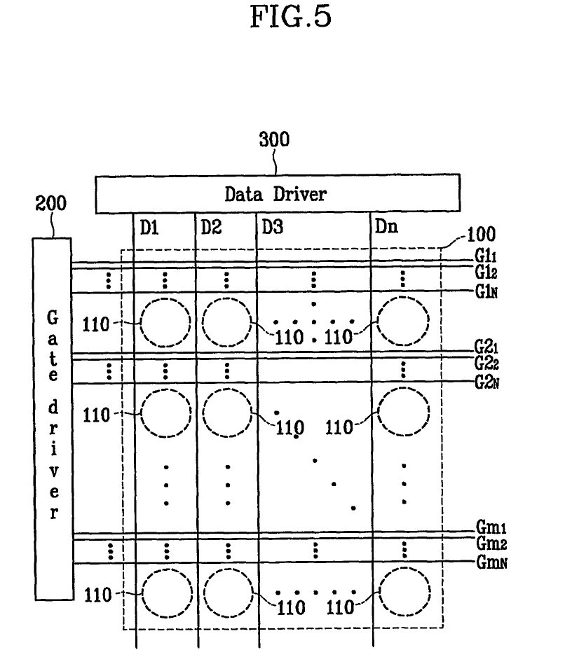

An embodiment of diving one gate line into a plurality of signal lines will be described in detail with reference to FIGS. 5 to 7.

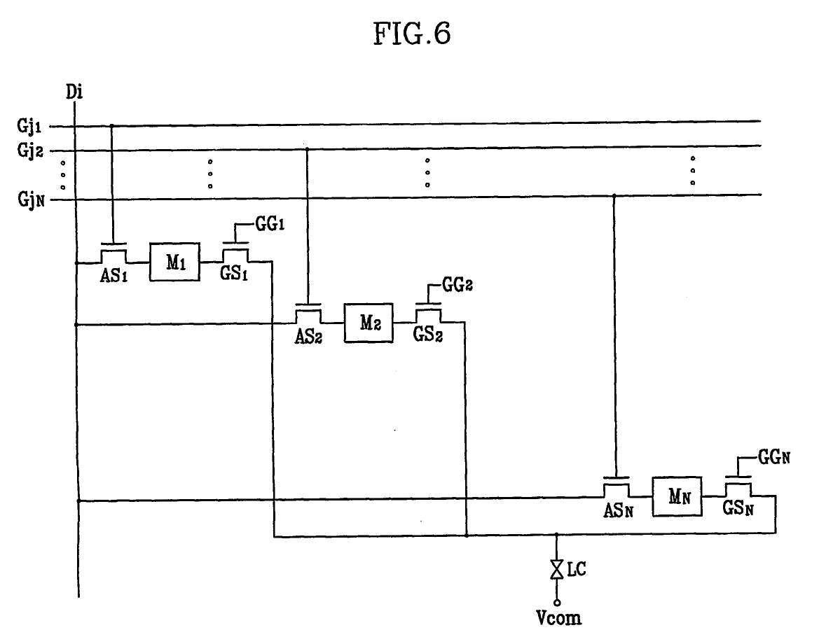

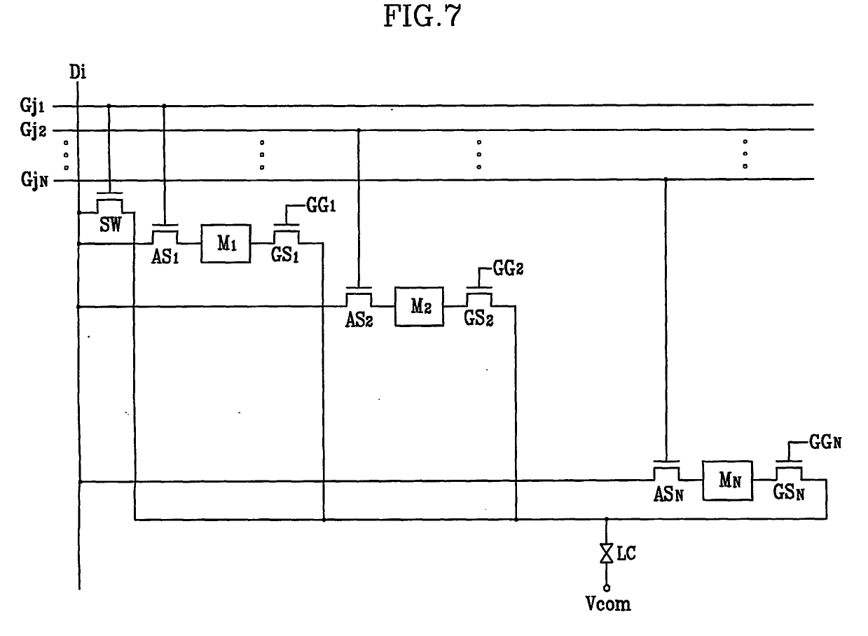

FIG. 5 shows an LCD according to a third embodiment of the present invention, and FIG. 6 and FIG. 7 show single pixel circuits of LCDs according to third and fourth embodiments of the present invention.

As shown in FIG. 5, an LCD according to a third embodiment of the present invention has substantially the same configuration as that according to the first embodiment except for a gate driver 200, a data driver 300, gate lines G1-Gm, and data lines D1-Dn.

In detail, the LCD according to the third embodiment includes a plurality of gate line groups G1-Gm, each gate line group Gj including a plurality of signal lines Gj₁-Gj_{N}. Instead, one data line Di does not include a plurality of signal lines unlike the first embodiment.

A pixel circuit 110 connected to an i-th data line Di and j-th gate lines Gj₁-Gj_{N} of the LCD according to the third embodiment of the present invention will be described in detail with reference to FIG. 6.

A pixel circuit 110 according to the third embodiment of the present invention includes a plurality of addressing switching elements AS₁-AS_{N} connected between the data line Di and a plurality of memory circuits M₁-M_{N} as shown in FIG. 6. The addressing switching elements AS₁-AS_{N} store digital data applied through the data line Di in the memory circuits M₁-M_{N} in response to the gate signals applied through the respective gate lines Gj₁-Gj_{N}. The storage of the data in the memory circuits M₁-M_{N} is performed during an addressing frame AF like the first embodiment

A plurality of gray switching elements GS₁-GS_{N} connected between the memory circuits M₁-M_{N} and the liquid crystal cell LC drive the liquid crystal cell LC with the data stored in the memory circuits M₁-M_{N} in response to driving signals GG₁-GG_{N} from an external device. The gray is determined by ratio of time for representing the white gray and time for displaying the black gray during the entire frame, like the first embodiment

As shown in FIG. 7, an LCD according to a fourth embodiment of the present invention has substantially the same configuration as that according to the third embodiment except that a pixel circuit 110 further includes an analog switching element SW.

In the fourth embodiment, the pixel circuit 110 connected to i-th data line Di and j-th gate line Gj according to the fourth embodiment will be described in detail. The pixel circuit 110 further includes an analog switching element SW connected between one (e.g., Gi₁) of signal lines Gi₁-Gi_{N}. and a liquid crystal cell LC.

In case of displaying still images, like the first embodiment of the present invention, the data are once stored in a plurality of memory circuits M₁-M_{N}, and thereafter, a plurality of gray switching elements GS₁-GS_{N} is driven to apply the data stored in the memory circuits M₁-M_{N} to the liquid crystal cell LC, thereby implementing the grays. In case of displaying moving images, a plurality of addressing switching elements AS₁-AS_{N} and the gray switching element GS₁-GS_{N} are turned off and the analog switching element SW is turned on to drive the liquid crystal cell LC with analog data voltage applied through the data line Di, thereby implementing the grays.

The first to the fourth embodiments of the present invention store the data in the memory circuits by dividing one data line into a plurality of signal lines or one gate line into a plurality of signal lines. However, one data line and one gate line are divided into a plurality signal lines for storing the data in the respective memory circuits.

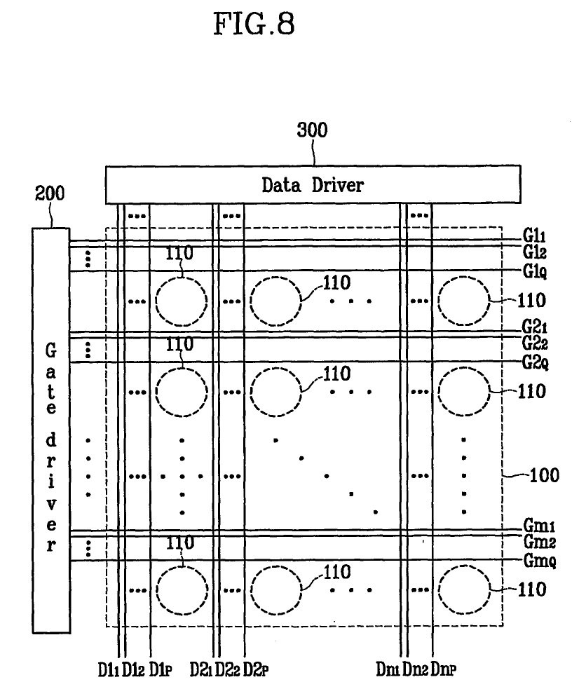

Now, an embodiment of dividing a data line and a gate line into a plurality of signal lines will be described with reference to FIGS. 8-10.

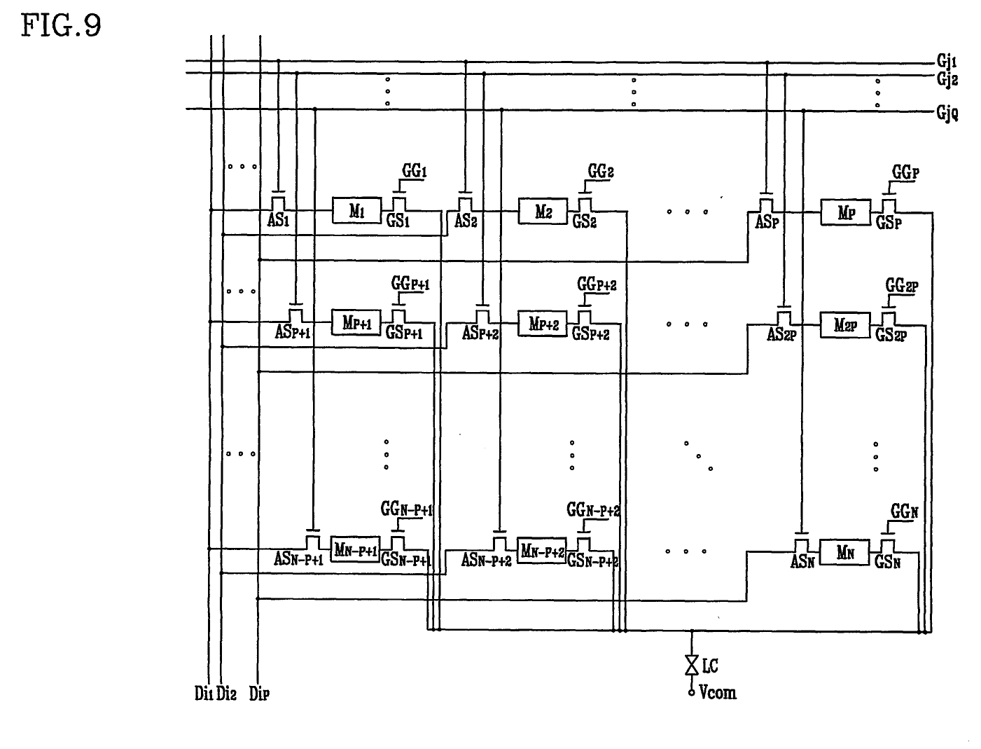

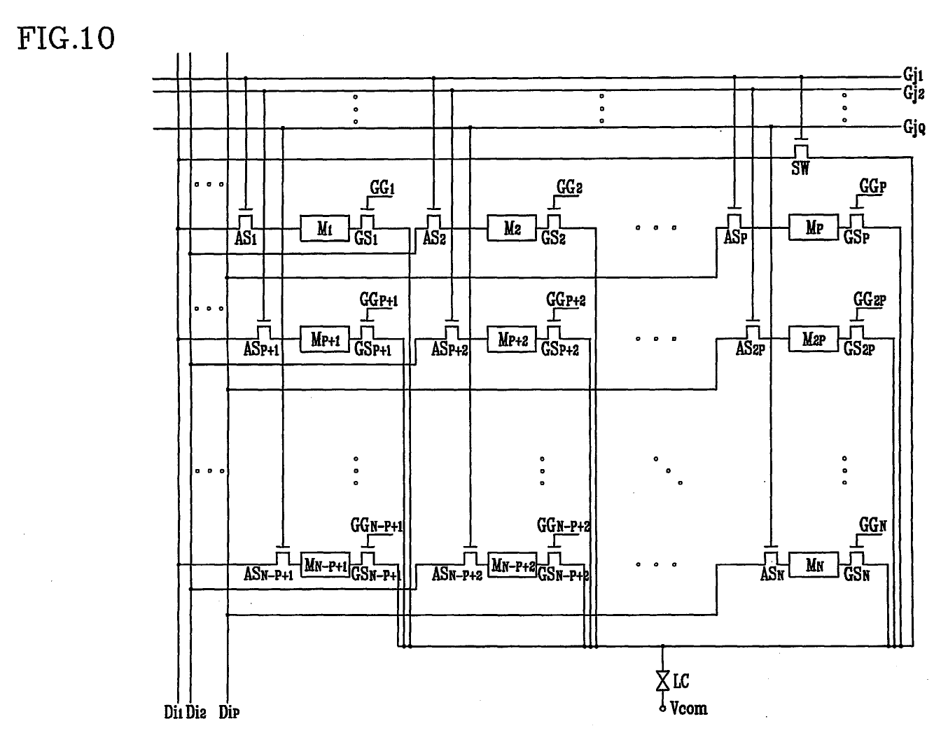

FIG. 8 shows an LCD according to a fifth embodiment of the present invention, and FIG. 9 and FIG. 10 show single pixel circuits of LCD according to fifth and sixth embodiments of the present invention.

As shown in FIG. 8, an LCD according to a fifth embodiment of the present invention has substantially the same configuration as that according to the first embodiment except a gate driver 200, a data driver 200 and 300, a plurality of gate lines G1-Gm, and a plurality of data lines D1-Dn.

In detail, in the LCD according to the fifth embodiment, one gate line group Gj and one data line group Di include a plurality of signal lines Gj₁-Gj_{P} and a plurality of signal lines Di₁-Di_{Q}, respectively. The multiple of the number (P) of the signal lines included in a gate line group and the number (Q) of the signal lines included in a data line group is preferably equal to or larger than the number of the memory circuits (P×Q≧N).

A pixel circuit 110 connected to an i-th data line Di₁-Di_{P} and j-th gate lines Gj₁-Gj_{Q} of the LCD according to the fifth embodiment of the present invention will be described in detail with reference to FIG. 9.

As shown in FIG. 9, a plurality of addressing switching elements AS₁-AS_{P} connected to memory circuits M₁-M_{P} are connected to data lines Di₁-Di_{P}, respectively, and store data applied through the data lines Di₁-Di_{P} into the memory circuits M₁-M_{P} in response to gate signals applied through the gate lines Gj₁. Similarly, a plurality of addressing switching elements AS_{P+1}-AS_{2P} are connected between the data lines Di₁-Di_{P} and the memory circuits M_{P+1}-M_{2P}, and store data applied through data lines Di₁-Di_{P} in the memory circuits M_{P+1}-M_{2P} in response to signals applied through gate lines Gj₂. In this way, it is possible to store the data in all of the memory circuits M₁-M_{P}, M_{P+1}-M_{2P}, . . . , M_{N}.

As described above, the storage of the data in the memory circuits is performed during interval of the addressing frame AF like the first embodiment. Since the step of driving a liquid crystal cell LC with the data stored in the memory circuits M₁-M_{N} is the same process as the first embodiment, the description thereof will be omitted.

A sixth embodiment of the present invention implements grays of still images by driving a liquid crystal cell using memory circuits, while implements grays of moving images by directly driving the liquid crystal cell without using memory circuits like the second and the fourth embodiments.

In detail, as shown in FIG. 10, a pixel circuit 110 of an LCD according to the sixth embodiment of the present invention further includes an analog switching element connected between one data line (e.g., Di₁) and a liquid crystal cell LC. In case of implementing grays of moving images, the analog switching element SW is driven according to a gate signal applied through one gate line (e.g., Gj₁) and directly drives the liquid crystal cell with data applied through the data line Di₁.

Although the first to the sixth embodiments store the data in the memory circuits regarding a predetermined time within one frame as an addressing frame AF, it is also possible to store data in the memory circuits regarding one or more frames as an addressing frame AF.

In addition, although LCDs are described as an example of flat panel displays, the present invention is not limited to this but is also applicable to flat panel displays of driving pixels in an active matrix type. The plat panel displays include all of the flat panel displays capable of implementing grays by average of time to drive display material, such as a FED and an electroluminescent display.

According to the present invention, it is possible to drive the flat panel display using the data stored in the memories without applying new data whenever driving the flat panel display when displaying still images. Therefore, it is possible to decrease the power consumption since there is no need of applying new data each time when displaying still images.

1. A flat panel display comprising:
a display panel provided with a plurality of pixels arranged in a matrix, one of the pixels including at least one first signal line extending in a direction, at least one second signal line crossing the first signal line, and a pixel circuit connected to the first signal line and the second signal line,
wherein the pixel circuit comprises:
a plurality storages storing data transmitted through the first signal line during an addressing time;
a plurality of first switching elements connected to the storages and transmitting the data from the first signal line to the storages in response to signals from the second signal line;
a display cell displaying images for the pixel according to the data stored in the storages; and
a plurality of second switching elements connected to the storages and driven in sequence during a frame to send the data stored in the storages to the display cell.

2. The flat panel display of claim 1, wherein the data stored in the storages has a first value making the display cell represent white gray and a second value making the display cell represent black gray.

3. The flat panel display of claim 1, wherein the pixel circuit further comprises a third switching element connected between one of the first signal lines and the display cell and transmitting data representing a variety of grays from the one of the first signal line to the display cell in response to a signal from the second signal line,
wherein the data stored in the storages has a first value making the display cell represent white gray and a second value making the display cell represent black gray.

4. The flat panel display of claim 1, wherein the first signal line includes a plurality of signal lines connected to the first switching elements.

5. The flat panel display of claim 1, wherein the first signal line includes a single signal line connected to the first switching elements, and the number of the at least one second signal line is equal to the number of the first switching elements.

6. The flat panel display of claim 1, wherein the first signal line and the second signal line include a plurality of signal lines, respectively, and the first switching elements correspond to one of the first signal lines and one of the second signal lines.

7. The flat panel display of claim 1, wherein the pixel circuit further comprises a plurality of inverting switching elements inverting the data stored in the storages to be applied to the display cell.

8. The flat panel display of claim 1, wherein the address time is a predetermined time shorter than one frame or equal to or longer than one frame.

9. A driving method of a flat panel display including a plurality of pixels having display cells, the method comprising:
a first step of storing data into a plurality of storages formed in the pixels during a predetermined time; and
a second step of implementing grays by dividing one frame or a portion of one frame into a plurality of sub-frames having different durations and sequentially driving the display cells with the data stored in the storages during the sub-frames.

10. The driving method of claim 9, wherein the address time is a portion of one frame or at least one frame.

11. The driving method of claim 9, wherein the second step drives the display cells by alternately applying the data stored in the storages and inverted data to the display cells.

12. The driving method of claim 9, wherein the data stored in the storages has a first value making the display cell represent white gray and a second value making the display cell represent black gray.

13. The driving method of claim 9, wherein the first step is performed such that when data representing still images are inputted, the data are stored in the storages, and when data representing moving images are inputted, the display cells are directly driven.

14. The driving method of claim 13, wherein the data representing the still images has a first value making the display cell represent white gray and a second value making the display cell represent black gray, and the data representing moving images has a value making the display cells represent a variety of grays.
